(19) United States
(12) Patent Application Publication
(10) Pub. No.: US 2005/0074827 A1
(43) Pub. Date: Apr. 7, 2005

(54) Methods for identifying modulators of quorum-sensing signaling in bacteria

(76) Inventors: Ute Muh, Iowa City, IA (US); Eric Robert Olson, Belmont, MA (US)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 10/894,710

(22) Filed: Jul. 19, 2004

Related U.S. Application Data
(60) Provisional application No. 60/488,591, filed on Jul. 18, 2003.

(51) Int. Cl. G01N 33/554; G01N 33/569; C12Q 1/18

(52) U.S. Cl. 435/7.32; 435/32

(57) ABSTRACT

The invention provides methods for identifying and analyzing modulators of quorum-sensing signaling in bacteria.

### CROSS-REFERENCE TO RELATED APPLICATIONS

This application claims the benefit of U.S. Provisional application No. 60/488,591, filed Jul. 18, 2003, which is hereby incorporated by reference in its entirety for all purposes.

### FIELD OF THE INVENTION

This invention relates generally to the field of molecular biology. More particularly, this invention relates to methods for identifying and analyzing modulators of quorum-sensing signaling in bacteria.

### BACKGROUND OF THE INVENTION

Bacteria communicate with each other to coordinate expression of specific genes in several ways, the best understood of these is designated quorum sensing. Quorum sensing enables a population of bacteria to sense its own numbers and to regulate gene expression according to population density. Cell-density-dependent regulation of genes in quorum sensing involves a freely diffusible molecule called autoinducer. Autoinducer is made in the cell by an endogenous synthase (designated LuxI in Vibrio fischeri) and when it reaches a high enough concentration it interacts with a cognate regulator (designated LuxR in V. fischeri) resulting in altered expression of specific genes.

Many bacteria have been shown to possess one or more quorum-sensing systems. These systems regulate a variety of physiological processes, including the expression of virulence genes and the formation of biofilms (Passador et al., Science 260: 1127-30 (1993); Davies et al., Science 280: 295-98 (1998)). Biofilms are an association of microorganisms, single or multiple species that grow attached to a surface and produce a slime layer that provides a protective environment. Typically, biofilms produce large amounts of extracellular polysaccharides.

In most natural settings, bacteria grow in biofilms. Biofilms also are associated with certain medical conditions. For example, biofilms of Pseudomonas aeruginosa have been isolated from medical implants, such as indwelling urethral venous or peritoneal catheters, and chronic P. aeruginosa infections in cystic fibrosis lungs are biofilms. In addition, bacterial biofilms interfere with industrial processes, where the formation of biofilms is often referred to as “biofouling.” Biofouling leads to material degradation, product contamination, mechanical blockage, and impedance of heat transfer in water-processing systems. Biofilms are also the primary cause of biological contamination of drinking water distribution systems due to growth on filtration devices.

Accordingly, there is a need to modulate quorum-sensing signaling in bacteria to interfere with the growth of biofilms, including biofilms of pathogenic bacteria. Assay methods have been developed to identify modulators of quorum-sensing signaling (e.g., International Publication No. WO 01/18248), but these methods are not sufficiently sensitive and they are not readily adaptable to high-throughput systems. Thus, there remains a need for methods for rapidly and efficiently identifying chemical entities that have the ability to modulate the quorum-sensing signaling pathway in bacteria.

### SUMMARY OF THE INVENTION

The present invention is based, at least in part, on the discovery that certain quorum-sensing-controlled promoters are surprisingly well-suited to high-throughput analysis. This discovery provided the capability to develop high-throughput methods to identify and analyze modulators of bacterial quorum-sensing. Such modulators are useful for controlling bacterial growth and gene expression and can be used for therapeutic treatment of bacterial infections particularly in immunocompromised subjects. They are also useful in treating disease states associated with biofilm development.

In some embodiments, the invention provides a method for identifying a modulator of bacterial quorum-sensing signaling comprising: (a) exposing a candidate compound to a culture of a bacterial strain comprising an optimal quorum-sensing-controlled promoter operably linked to a reporter gene; (b) measuring a first amount of a product of said reporter gene in said culture; and (c) comparing said first amount to a second amount of said product of said reporter gene, said second amount measured in the absence of said candidate compound.

In some embodiments, the invention provides a method for determining whether a modulator of bacterial quorum-sensing signaling acts downstream of autoinducer synthesis comprising: (a) exposing said modulator to a culture of a bacterial strain comprising an optimal quorum-sensing-controlled promoter operably linked to a reporter gene, wherein said bacterial strain does not produce autoinducer capable of inducing said promoter; (b) exposing said culture to an autoinducer capable of inducing said promoter; (c) measuring a first amount of a product of said reporter gene in said culture; and (d) comparing said first amount to a second amount of said product of said reporter gene, said second amount measured in the absence of said candidate compound.

In some embodiments, the invention provides a method for identifying a modulator of bacterial quorum-sensing signaling comprising: (a) exposing a candidate compound to a culture of a bacterial strain comprising an optimal quorum-sensing-controlled promoter operably linked to a reporter gene, wherein said bacterial strain does not produce autoinducer capable of inducing said promoter; (b) exposing said culture to an autoinducer capable of inducing said promoter; (c) measuring a first amount of a product of said reporter gene in said culture; and (d) comparing said first amount to a second amount of said product of said reporter gene, said second amount measured in the absence of said candidate compound.

In some embodiments, the invention uses a bacterial strain selected from the group consisting of: a Pseudomonas aeruginosa strain, an Escherichia coli strain, a Salmonella typhimurium strain, and a Shigella flexneri strain. In some embodiments, the bacterial strain is a P. aeruginosa strain.

In some embodiments, the invention uses a promoter regulated by LasR, RhlR or both. In some embodiments, the promoter is regulated by LasR. In some embodiments, the promoter is from the rsaL gene. In some embodiments, the bacterial strain lacks LasI function.

In some embodiments, the optimal quorum-sensing-controlled promoter and the reporter gene are in a vector. In some embodiments, the vector is a plasmid.

In some embodiments, the bacterial strain used in the methods of the invention has a functional drug efflux system. In some embodiments, the bacterial strain is drug-resistant. In some embodiments, the bacterial strain is resistant to an antibiotic selected from the group consisting of: a fluoroquinolone antibiotic, a β-lactam antibiotic, an aminoglycoside antibiotic, and a macrolide antibiotic.

In some embodiments, the reporter gene used in the methods of the invention is detectable by optical means. In some embodiments of the invention the reporter gene is detectable by fluorescence. In some embodiments, the reporter gene is selected from the group consisting of: lacZ, gusA, cat, lux and gfp. In some embodiments, the reporter gene is gfp.

In some embodiments, the methods of the invention are performed in a high-throughput format. In some embodiments, the high-throughput format uses a 96-well plate or a 3456 NanoWell™ plate.

Unless otherwise defined, all technical and scientific terms used herein have the same meaning as commonly understood by one of ordinary skill in the art to which the invention pertains. In case of conflict, the present specification, including definitions, will control. All publications, patents and other references mentioned herein are incorporated by reference in their entireties for all purposes.

### BRIEF DESCRIPTION OF THE DRAWINGS

FIG. 1 shows the induction of yfp measured by relative fluorescence units in MW1.pUM15 grown in the absence (open circles) and presence (closed circles) of 0.9 mM 3-oxo-C12-HSL.

FIG. 2 shows titration of yfp-expression in MW1.pUM15 against signal concentration. The data were fitted in first approximation with the Michaelis-Menten equation, indicating that half-maximal induction was reached at 0.2 mM 3-oxo-C12-HSL.

FIG. 3 graphs induction of yfp in PAO1.pUM15/cell relative to OD₆₀₀.

FIG. 4 shows the effect of an inhibitor on expression in the complete signaling assay. The cells, PAO1.pUM15, were grown with no compound added (closed circles), in the presence of 0.1% DMSO (open circles) and 100 mM inhibitor (closed triangles).

FIG. 5 shows the effect of an inhibitor on expression in the signal reception assay. The cells, MW1.pUM15, were grown with signal, no inhibitor, no DMSO (closed circles); signal, no inhibitor, 0.1% DMSO (open circles); signal, 100 mM inhibitor (closed triangles); no signal, no inhibitor, no DMSO (open triangles).

### DETAILED DESCRIPTION OF THE INVENTION

The present invention provides methods of identifying and analyzing modulators of bacterial quorum-sensing signaling.

In some embodiments, the invention provides methods for identifying modulators of bacterial quorum-sensing signaling comprising exposing cultures of a bacterial strain comprising a quorum-sensing-controlled promoter operably linked to a reporter gene to candidate compounds and comparing the expression of the promoter to expression in the absence of the candidate compounds. In this method, a decrease in the amount of the product of the reporter gene indicates that the compound inhibits bacterial quorum-sensing signaling, and an increase indicates that the compound promotes bacterial quorum-sensing signaling.

In some embodiments, the invention provides a method of identifying a modulator of bacterial quorum-sensing signaling, wherein the bacterial strain comprising the quorum-sensing-controlled promoter operably linked to a reporter gene does not produce autoinducer. Accordingly, this method will not identify modulators that modulate autoinducer synthesis. In addition, in these embodiments, autoinducer may be added to the culture at different concentrations, which can be tailored to make the assay more sensitive to modulators acting downstream of the synthase. For example, autoinducers may be added at a concentration such that expression of the promoter is at least 10%, at least 25%, at 50% at least 75%, at least 95% or 100% of its highest naturally induced level. In this method, a decrease in the amount of the product of the reporter gene indicates that the compound inhibits bacterial quorum-sensing signaling, and an increase indicates that the compound promotes bacterial quorum-sensing signaling.

In some embodiments, the invention provides a method for determining whether a modulator acts downstream of autoinducer synthesis, wherein the bacterial strain comprising the quorum-sensing-controlled promoter operably linked to a reporter gene does not produce autoinducer. A modulator that acts upstream of autoinducer synthesis would not modulate bacterial quorum-sensing signaling in this method and, conversely, a modulator that acts downstream of autoinducer synthesis would modulate bacterial quorum-sensing signaling.

As used herein, “bacterial quorum-sensing signaling” is the signaling mediated by a bacterial cell-to cell communication system which enables population-density-dependent regulation of gene expression. Such systems regulate genes involved in a wide variety of phenotypes. In some cases, one bacterial strain will have multiple quorum-sensing signaling system, which may regulate the same or distinct sets of genes. For an overview of bacterial quorum-sensing signaling see, e.g. Michiko & Bassler, Proc. Natl. Acad. Sci. USA 100: 14549-54 (2003); Smith & Iglewski, Curr. Opin. Microbiol. 6: 56-60 (2003)). Table 1 provides a list of exemplary bacteria that exhibit bacterial quorum-sensing signaling.

TABLE 1
Exemplary list of bacteria with quorum-sensing
signaling system(s).

| | Homologs to | | Phenotype(s) associated with quorum- |
|---|---|---|---|
| Bacterial species | LuxR/LuxI | Major autoinducer molecule | sensing signaling |
| Vibrio fischeri | LuxR/LuxI | 3-oxo-C6-HSL | Bioluminescence |
| Aeromonas hydrophila | AhyR/AhyI | C4-homoserine lactone (HSL) | Extracellular protease, biofilm formation |
| Aeromonas salmonicida | AsaR/AsaI | C4-HSL | Extracellular protease |
| Agrobacterium tumefaciens | TraR/TraI | 3-oxo-C8-HSL | Conjugation |
| Burkholderia cepacia | CepR/CepI | C8-HSL | Protease, siderophore |
| Chromobacterium violaceum | CviR/CviI | C6-HSL | Antibiotics, violacein, exoenzymes, cyanide |
| Erwinia carotovora ssp. | CarR, ExpR/ | 3-oxo-C6-HSL | Carbapenem antibiotic, exoenzymes |
| carotovora | ExpI | | |
| Erwinia chrysanthemi | ExpR/ExpI | 3-oxo-C6-HSL | Pectinases |
| Pantoea stewartii | EsaR/EsaI | 3-oxo-C6-HSL | Exopolysaccharide |
| Pseudomonas aeruginosa | LasR/LasI | 3-oxo-C12-HSL | Exoenzymes, Xcp, biofilm formation, |
| | | | RhlR, cell-cell spacing |
| Pseudomonas aeruginosa | RhlR/RhlI | C4-HSL | Exoenzymes, cyanide, |
| | | | RpoS, lectins, pyocyanin, |
| | | | rhamnolipid, type 4 pili |
| Pseudomonas aureofaciens | PhzR/PhzI | C6-HSL | Phenazine antibiotic |
| Serratia liquefaciens | SwrR/SwrI | C4-HSL | Swarming, protease |
| Xenorhabdus nematophilus | Unknown | 3-hydroxy-C4-HSL or an agonist | Virulence, bacterial lipase |
| Yersinia pseudotuberculosis | YpsR/YpsI | 3-oxo-C6-HSL | Motility, clumping |

As used herein, an “autoinducer” is a molecule that leads to expression of quorum-sensing-controlled promoters when present at a sufficiently high concentration. Three chemical types of molecules have been identified: N-acyl-L-homoserine lactones, peptides, and AI-2 like molecules of Vibrio species, an example being a furanosyl borate diester. Autoinducers can be obtained, e.g., by purifying them from bacterial cultures or they can be chemically synthesized.

As used herein, a “modulator of bacterial quorum-sensing signaling” includes compounds that alter quorum-sensing signaling in any way, including, e.g., increasing, decreasing, blocking, and delaying. Such a modulator can act at any step in the bacterial quorum-sensing signaling pathway. For example, a modulator may influence the ability of a bacterial strain to synthesize an autoinducer or a modulator may act downstream of the autoinducer synthesis, e.g., it may influence the ability of a bacterial cell to perceive the presence of autoinducer and respond through the modulation of expression of genes that are regulated by that system. A modulator may act downstream of autoinducer synthesis by, e.g., completely eliminating autoinducer perception or increasing the concentration of autoinducer necessary to modulate quorum-sensing-controlled promoter expression.

The methods of the invention involve a culture of a bacterial strain. As used herein, a “culture of a bacterial strain” includes bacteria grown in liquid media as well as in or on solid or semisolid media.

The methods of the invention may be performed with any bacterial strain. Typically, the methods are performed with a Gram-negative bacterial strain, generally with a Gram-negative strain from a species selected from: Pseudomonas aeruginosa, Escherichia coli, Salmonella typhimurium, and Shigella flexneri.

As used herein, an “optimal quorum-sensing-controlled promoter” is a quorum-sensing-controlled promoter that has two specific properties. These properties make these promoters surprisingly useful in the methods of the invention.

First, the highest level of expression of an optimal quorum-sensing-controlled promoter in the presence of autoinducer is at least 50-fold greater than the expression of the gene in the bacterial cell that encodes the autoinducer synthase. For example, the highest level of expression in the presence of 3-oxo-C12-HSL of an optimal quorum-sensing-controlled promoter from P. aeruginosa would be at least 50-fold greater than the expression from the lasI promoter in the presence of the same concentration of 3-oxo-C12-HSL. In some embodiments, the optimal quorum-sensing-controlled promoter exhibits more than at least 50-fold greater expression relative to expression of the gene in the bacterial cell that encodes the autoinducer synthase, e.g., 60-fold, 70-fold, 80-fold, 90-fold, or 100-fold.

Second, the highest level of expression of an optimal quorum-sensing-controlled promoter in the presence of an autoinducer is at least 5-fold greater than the expression of the promoter in the absence of the autoinducer. For example, the highest level of expression in the presence of 3-oxo-C12-HSL of an optimal quorum-sensing-controlled promoter from P. aeruginosa would be at least 5-fold greater than its expression in the absence of 3-oxo-C12-HSL. In some embodiments, the optimal quorum-sensing-controlled promoter exhibits more than at least 5-fold greater expression, e.g., 10-fold, 15-fold, 20-fold, 25-fold, or 30-fold.

One of ordinary skill in the art will recognize that only a proportion of quorum-sensing-controlled promoters in a bacterial strain will be optimal quorum-sensing-controlled promoter. An examplary optimal quorum-sensing-controlled promoter useful in the methods of the invention is the promoter from the P. aeruginosa rsaL gene.

As used herein, a “reporter gene” is a gene that encodes a detectable product. The detectable product is also generally quantifiable. For example, expression of a reporter gene can be detected and quantified by measuring levels of its RNA transcript, levels of its encoded protein, or activity of its encoded protein. Reporter genes for use in the invention include, e.g., the endogenously linked gene as well as heterologous sequences including, e.g., β-galactosidase (lacZ), β-glucuronidase (gusA), chloramphenical acetyl transferase (cat), luciferase (luc, luxA), and green fluorescent protein (gfp). As used herein, the use of the designation “gfp” is understood to include both wild-type gfp as well as its many color-shifted and/or stabilized variants (e.g., blue-, cyan- and yellow-shifted variants (bfp, cfp, yfp)).

The detectable product of a reporter gene may be measured using any suitable means. The selection of suitable measuring means is within the ordinary skill in the art. For example, β-galactosidase and β-glucuronidase are typically measured using specific antibodies or an enzymatic substrate that generates color when cleaved by β-galactosidase or β-glucuronidase, respectively. Green fluorescent protein and its variants are generally measured by exciting them with one wavelength of light and detecting emission at a second wavelength. One of ordinary skill in the art would recognize that different measurement means may be selected depending on various factors including, e.g., sensitivity, speed, and cost.

The methods of the invention use an optimal quorum-sensing-controlled promoter operably linked to a reporter gene. The promoter operably linked to the reporter gene may be present in the genome of the bacterial cell or in an extrachromosomal element such as a plasmid, viral vector, or a BAC. These vectors typically also contain a marker gene for the selection of bacterial cells containing them.

The methods of the invention are typically performed in a high-throughput screening format. In fact, because of their particular properties as described above, optimal quorum-sensing-controlled promoters are surprisingly better suited to high-throughput screening methods. For high-throughput screening, reporter genes are typically used that are detectable by optical means, especially by fluorescence (e.g., gfp or one of its many color-shifted and/or stabilized variants),. Many systems for high-throughput screening are known in the art. For example, systems useful for performing the methods of the invention in a high-throughput format are described in U.S. Pat. Nos. 5,985,214; 6,472,218; 6,468,800; 6,063, 338; 6,232,114; 6,229,603; 5,910,287; 6,349,160; 6,254,833; 6,171,780; 6,517,781;6,296,8 11; 6,426,050; 6,372,185; 6,448,089; 6,586,257; U.S. patent application Nos. US20020012611A1; US20020001075A1; US20020155617A1; US20010055814A1; US20030039591A1; US20020119077A1; US20020192116A1; and International Patent Publication Nos. WO 98/55231; WO 00/04366; WO 99/42608; WO 00/33961; and WO 01/27635.

In some embodiments, the methods of the invention are performed using a bacterial strain with a functional drug efflux system. Such a bacterial strain may be advantageously used in the methods of the invention to identify modulators that can either bypass or overwhelm the bacterial detoxification system.

In some embodiments, the methods of the invention are performed using a drug-resistant bacterial strain. As used herein, a bacterial strain is drug-resistant if the minimal inhibitory concentration (MIC) for a drug in the resistant strain is at least four-fold greater than the MIC for the same drug in a National Committee for Clinical Laboratory Standards (NCCLS) reference strain of the same bacterial species. Bacteria can acquire resistance to a particular drug in a variety of ways, including, e.g., acquiring a gene that produces an enzyme capable of breaking down a particular drug (e.g., resistance to β-lactam antibiotics), mutations that increase drug efflux (e.g., resistance to macrolide antibiotics), alterations in molecules which are the targets of the drugs so that interaction of the drug with its target is reduced (e.g., resistance to rifampicin), and acquisition of new genes that bypass the action of the antibiotic (e.g., resistance to vancomycin). For example, a method of the invention may be performed using a bacterial strain that is resistant to a fluoroquinolone antibiotic, a β-lactam antibiotic, an aminoglycoside antibiotic, or a macrolide antibiotic.

Although methods and materials similar or equivalent to those described herein can be used in the practice or testing of the invention, specific methods and materials that may be used in the invention are described below. While the materials, methods and examples exhibit some embodiments of the invention, they are illustrative only, and are not intended to limit the full scope of the invention. Other features and advantages of the invention will be apparent from the description and from the claims.

### EXAMPLES

### Example 1

### Development of a High-Throughput Assay System to Monitor the Quorum-Sensing Signaling Pathway

Strain Construction. We tested whether the P. aeruginosa quorum-sensing-controlled promoter for the rsaL gene (PrsaL) can be used in high-throughput screening to identify modulators of quorum-sensing signaling. We constructed a plasmid (designated pUM15) with yfp under control of the P. aeruginosa LasR-controlled promoter PrsaL and introduced the plasmid into P. aeruginosa wild-type strain PAO1 (designated PAO1.pUM15). In PAO1.pUM15, the expression of yfp is dependent on the endogenously synthesized autoinducer, 3-oxo-C12-HSL. Thus, fluorescence will be altered if a test compound modulates any aspect of signaling, including, e.g., signal synthesis, stability, reception, or expression of LasR-dependent genes. Accordingly, we designated this assay the “Complete Signaling Assay” or CSA. We also introduced pUML15 into a P. aeruginosa strain with null alleles of the signal synthase genes lasI and rhlI (MW1). In the experiments in this Example, MW1 served as a negative control, because it is incapable of synthesizing 3-oxo-C12-HSL.

Assay System. We inoculated a culture of LB with 300 μg/mL carbenicillin with PAO1.pUM15 from an LB-agar, carbenicillin plate (the colonies on the plates were always struck directly from a freezer stock and had been on the plates for less than 5 days; LB broth was 10 g tryptone, 5 g yeast extract, and 4 g NaCl per 1 L water; carbenicillin was maintained as a 300 mg/mL filter-sterilized stock in water, which was stored at −20° C.). We allowed the culture to grow overnight at 37° C. with shaking at 250 rpm. The next morning, we washed the cells twice with LB and subcultured the washed cells at an A₆₀₀ of 0.05-0.1 (1 cm pathlength) into LB supplemented with 300 μg/mL carbenicillin. In some experiments, we also tested an initial inoculum at lower density cells, but aside from the fact that the plates needed to be incubated for longer periods of time (which in some instances led to clumping), we did notobserve a significant variability in the performance of the cells. We pipeted 50 μL of this subculture into each well of an untreated 96-well plate. When added, the test compound in DMSO and/or 3-oxo-C12-HSL in ethyl acetate were placed in individual wells before bacterial addition and dried in a laminar flow hood or under a stream of sterile air/N₂. We also tested treated 96-well plates, such as those with non-binding or tissue culture treated polymer, and observed that the results did not differ significantly from untreated plates. We incubated the plates at 37° C., for 8-12 h in a sealed, humidified container, at which time maximal induction of fluorescence was observed (results with stationary plates and those that were shaken did not differ significantly).

Measurement of Reporter Gene Product. We measured fluorescence with an excitation filter at 485 nm and an emission filter at 535 nm in a Tecan SPECTRAFluor Plus plate reader. We found that under the conditions of the assay, reading the fluorescence from the bottom of the well consistently led to a 2-fold increase in the dynamic range compared to a top read. The signal was reduced in the presence of DMSO and the best results were obtained at DMSO concentrations of 1% or less.

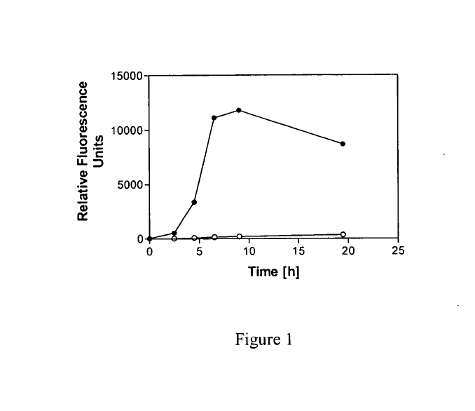

Expression Depends on Autoinducer. Our data confirmed that regulation of yfp expression from the pUM15 plasmid is dependent on 3-oxo-C12-HSL. We detected very little fluorescence in MW1.pUM15 culture grown in the absence of signal molecule (FIG. 1). Addition of 3-oxo-C12-HSL restored expression of yfp in pMW1.pUM15, confirming that yfp in pUM15 is dependent on the autoinducer 3-oxo-C 12-HSL (FIG. 1).

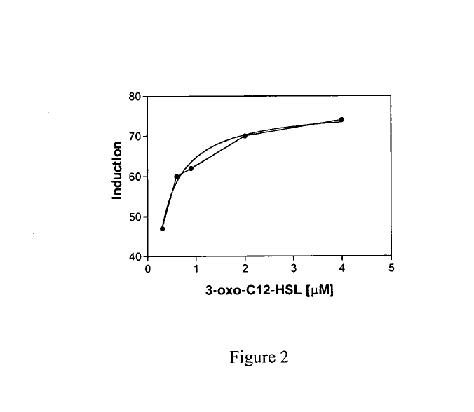

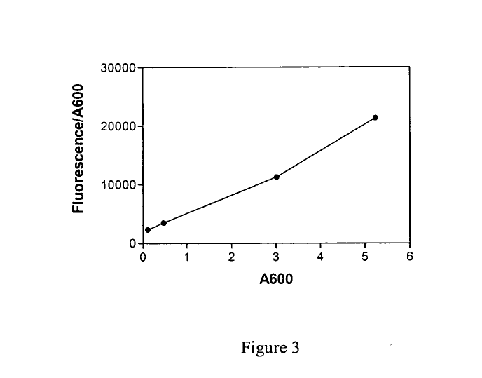

The Assay Mimics Naturally Occurring Quorum-Sensing Signaling. We assayed expression at a range of concentrations of autoinducer. We observed that fluorescence in MW1.pUM15 is half-maximal at 200±100 nM 3-oxo-C12-HSL (FIG. 2). These results were consistent with an observed half-maximal saturation of 200 nM using a cell-based binding assay, 3-oxo-C12-HSL (Passador et al., J. Bacteriol. 178: 5995-6000 (1996)) and indicated that our assay system is representative of the naturally occurring quorum-sensing signaling system. We also confirmed that expression of yfp is quorum-sensing dependent by graphing fluorescence per cell against cell density (FIG. 3). If expression of yfp were constitutive, the graph would be a horizontal line. Instead, fluorescence per cell increased at higher density, confirming that the PrsaL-yfp construct in pMW15 is quorum-sensing controlled.

The Assay Is Statistically Robust. Typically, screen validation consists of determining statistical robustness (termed “assay window”) of an assay and then verifying the reproducibility and robustness in a mock screen using positive control compounds randomly spiked into the assay plate. Other important screen criteria, such as the performance of the assay over an experimental run are also assessed. We tested several of these parameters in the assay protocol, which included a final incubation volume of 50 μl in 96-well plates.

The statistical robustness of the assay was tested by running three plates on three days with 8 wells containing PAO1.pUM15 and 8 wells containing MW1.pUM15. The screening window parameter over the three days was calculated as:
[3*(SD signal+SD baseline)/(signal−baseline)]

This statistical parameter takes into account the separation between positive and negative controls and the variability of the data to give a measure of the reliability of separating hits from the background. A value of less than one insures an acceptably low rate of false-negative and false-positive wells. The results from the three-day experiments are summarized in Table 2.

TABLE 2
Summary of Data on Statistical Robustness of CSA

| | Day 1 | Day 2 | Day 2 | Day 3 | Day 3 |
|---|---|---|---|---|---|
| Read | top | top | bottom | top | bottom |
| AVG Signal | 13307 | 15710 | 45385 | 14201 | 39376 |
| SD Signal | 913 | 1160 | 4562 | 1637 | 3277 |
| % CV Signal | 6.86 | 7.38 | 10.1 | 11.5 | 8.32 |
| AVG Base | 242 | 235 | 679 | 155 | 517 |
| SD Base | 11 | 20 | 62 | 8 | 41 |
| % CV Base | 4.53 | 8.5 | 9.13 | 5.16 | 7.93 |
| Window | 0.212 | 0.229 | 0.316 | 0.351 | 0.256 |

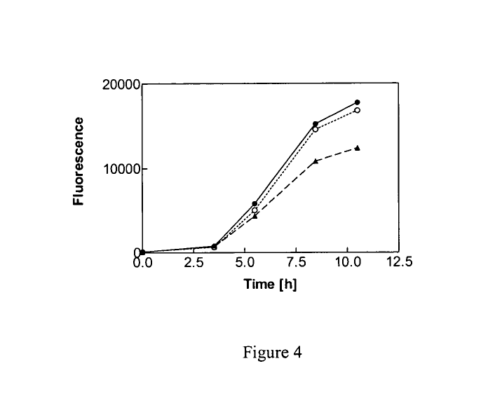

The Assay Accurately Reports the Activity of a Known Modulator. We tested an inhibitor of quorum-sensing signal reception that was identified in another screen. Under the assay conditions described here, addition of 100 μM of the inhibitor led to an approximately 25% reduction of signal (FIG. 4).

Adaptation of the Assay to 3456-Well Format. The assay was performed in a volume of 2 μL in 3456 NanoWell™ plates. Cells were prepared as described above. Plate additions were made with an FRD single-tip dispenser built at Aurora Biosciences Corp. Plates were incubated at 37° C. under humidified conditions. Fluorescence was measured from the bottom with the Aurora NanoPlate Reader. The assay window remained robust from 12 to 24 hours.

TABLE 3
CSA Results for 3456-Well Plates

| | Day 1 | Day 2 | Day 3 |
|---|---|---|---|
| AVG Signal | 4.04 | 3.82 | 3.84 |
| SD Signal | 0.109 | 0.195 | 0.272 |
| % CV Signal | 2.7 | 5.1 | 7.1 |
| AVG Base | 0.058 | 0.080 | 0.053 |
| SD Base | 0.004 | 0.003 | 0.006 |
| % CV Base | 7.0 | 4.3 | 11.0 |
| Window | 0.085 | 0.159 | 0.221 |

### Example 2

### Adaptation of the Assay to Detect Modulators of Signal Reception

We adapted the assay system described in Example 1 to identify only modulators that act downstream of autoinducer synthesis by performing the assay on MW1.pUM15. Since this strain lacks the lasI gene, which encodes the LasI signal synthase, induction of yfp expression requires addition of the 3-oxo-C12-HSL signaling molecule to the media. Thus, if a test compound modulates any aspect of signal reception, such as signal binding or transcriptional activation, fluorescence will be altered, but any modulator that acts on autoinducer synthesis will have no effect in the assay. Accordingly, we designated this assay the “Signal Reception Assay” or SRA.

Assay System. The assay system is that described in Example 1 with a few modifications. We inoculated a culture of LB, 50 mM MOPS pH 7.0, 300 μg/mL carbenicillin with MW1.pUM15 from an LB-agar carbenicillin plate (as before, the colonies on the plates were always struck directly from a freezer stock and had been on the plates for less than 5 days). We allowed the culture to grow overnight at 37° C. with shaking at 250 rpm (final OD₆₀₀ was no higher than 1.5 to 2). The next morning, we subcultured the cells at an A₆₀₀ of 0.05 (1 cm pathlength) into LB, 50 mM MOPS pH 7.0, 300 μg/mL carbenicillin and allowed the cells to grow for 60-120 minutes at 37° C. with shaking at 250 rpm. We pipeted 50 μL of this subculture into each well of an untreated 96-well plate. When added, the test compound in DMSO and/or 3-oxo-C12-HSL in ethyl acetate were placed in individual wells before bacterial addition and dried in a laminar flow hood or under a stream of sterile air/N₂. We incubated the plates and assayed fluorescence as described in Example 1.

SRA Is Statistically Robust. We tested the statistical robustness of the assay by running three plates on three days with 8 wells containing MW1.pUM15 and 0.3 mM 3-oxo-C12-HSL and 8 wells containing MW1.pUM15 without autoinducer. The screening window parameter over the three days was calculated as in Example 1. The results from the three-day experiments are summarized in Table 4.

TABLE 4
Summary of Data on Statistical Robustness of SRA

| | Day 1 | Day 2 | Day 2 | Day 3 | Day 3 |
|---|---|---|---|---|---|
| Read | top | top | bottom | top | bottom |
| AVG Signal | 4284 | 5250 | 13013 | 4273 | 11285 |
| SD Signal | 391 | 906 | 2589 | 645 | 1420 |
| % CV Signal | 9.13 | 17.3 | 19.9 | 15.1 | 12.6 |
| AVG Base | 184 | 144 | 431 | 140 | 464 |
| SD Base | 5 | 11 | 41 | 8 | 24 |
| % CV Base | 2.70 | 7.63 | 9.51 | 5.71 | 5.17 |

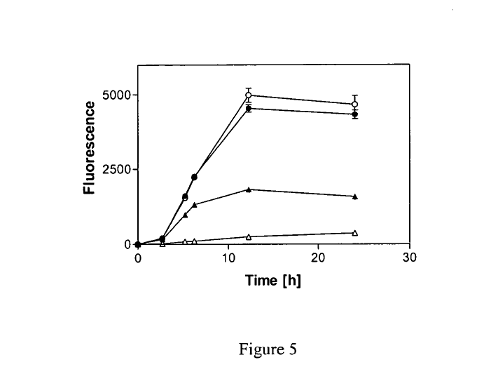

SRA Accurately Reports the Activity of a Known Modulator. We tested the inhibitor of quorum-sensing signaling used in Example 1 in the Signal Reception Assay. Under these assay conditions, addition of 100 μM of the inhibitor led to an approximately 60% reduction of signal (FIG. 5).

SRA in 3456-Well Format. SRA also was performed in a volume of 2 μL in 3456 NanoWell™ plates as described for CSA in Example 1. Cells were prepared as described above for 96-well plates. As before, we observed that the assay window remained robust from 12 to 24 hours.

TABLE 5
SRA Results for 3456-Well Plates

| | Day 1 | Day 2 | Day 3 |
|---|---|---|---|
| AVG Signal | 3.28 | 2.59 | 3.03 |
| SD Signal | 0.093 | 0.184 | 0.155 |
| % CV Signal | 2.8 | 7.2 | 5.2 |
| AVG Base | 0.109 | 0.075 | 0.084 |
| SD Base | 0.005 | 0.010 | 0.006 |
| % CV Base | 4.3 | 14.0 | 6.7 |
| Window | 0.092 | 0.232 | 0.164 |

### OTHER EMBODIMENTS

Other embodiments are within the following claims.

1. A method for identifying a modulator of bacterial quorum-sensing signaling comprising:
a) exposing a candidate compound to a culture of a bacterial strain comprising an optimal quorum-sensing-controlled promoter operably linked to a reporter gene;
b) measuring a first amount of a product of said reporter gene in said culture; and
c) comparing said first amount to a second amount of said product of said reporter gene, said second amount measured in the absence of said candidate compound.

2. The method according to claim 1, wherein said bacterial strain is selected from the group consisting of: a Pseudomonas aeruginosa strain, an Escherichia coli strain, a Salmonella typhimurium strain, and a Shigella flexneri strain.

3. The method according to claim 2, wherein said bacterial strain is a P. aeruginosa strain.

4. The method according to claim 3, wherein said promoter is regulated by LasR, RhlR or both.

5. The method according to claim 4, wherein said promoter is regulated by LasR.

6. The method according to claim 5, wherein said promoter is from the rsaL gene.

7. The method according to claim 1, wherein said promoter and said reporter gene are in a vector.

8. The method according to claim 7, wherein said vector is a plasmid.

9. The method according to claim 1, wherein said bacterial strain has a functional drug efflux system.

10. The method according to claim 1, wherein said bacterial strain is drug-resistant.

11. The method according to claim 10, wherein said bacterial strain is resistant to an antibiotic selected from the group consisting of: a fluoroquinolone antibiotic, a β-lactam antibiotic, an aminoglycoside antibiotic, and a macrolide antibiotic.

12. The method according to claim 1, wherein said reporter gene is detectable by optical means.

13. The method according to claim 12, wherein said reporter gene is selected from the group consisting of: lacZ, gusA, cat, lux and gfp.

14. The method according to claim 12, wherein said reporter gene is detectable by fluorescence.

15. The method according to claim 14, wherein said reporter gene is gfp.

16. The method according to claim 12, wherein said method is performed in a high-throughput format.

17. The method according to claim 16, wherein said high-throughput format uses a 96-well plate or a 3456 NanoWell™ plate.

18. A method for determining whether a modulator of bacterial quorum-sensing signaling acts downstream of autoinducer synthesis comprising:
a) exposing said modulator to a culture of a bacterial strain comprising an optimal quorum-sensing-controlled promoter operably linked to a reporter gene, wherein said bacterial strain does not produce autoinducer capable of inducing said promoter;
b) exposing said culture to an autoinducer capable of inducing said promoter;
c) measuring a first amount of a product of said reporter gene in said culture; and
d) comparing said first amount to a second amount of said product of said reporter gene, said second amount measured in the absence of said candidate compound.

19. A method for identifying a modulator of bacterial quorum-sensing signaling comprising:
a) exposing a candidate compound to a culture of a bacterial strain comprising an optimal quorum-sensing-controlled promoter operably linked to a reporter gene, wherein said bacterial strain does not produce autoinducer capable of inducing said promoter;
b) exposing said culture to an autoinducer capable of inducing said promoter;
c) measuring a first amount of a product of said reporter gene in said culture; and
d) comparing said first amount to a second amount of said product of said reporter gene, said second amount measured in the absence of said candidate compound.

20. The method according to claim 18 or 19, wherein said bacterial strain is selected from the group consisting of: a Pseudomonas aeruginosa strain, an Escherichia coli strain, a Salmonella typhimurium strain, and a Shigella flexneri strain.

21. The method according to claim 20, wherein said bacterial strain is a P. aeruginosa strain.

22. The method according to claim 20, wherein said promoter is regulated by LasR, RhlR or both.

23. The method according to claim 22, wherein said promoter is regulated by LasR.

24. The method according to claim 23, wherein said P. aeruginosa strain lacks LasI function.

25. The method according to claim 23, wherein said promoter is from the rsaL gene.

26. The method according to claim 18 or 19, wherein said promoter and said reporter gene are in a vector.

27. The method according to claim 26, wherein said vector is a plasmid.

28. The method according to claim 18 or 19, wherein said bacterial strain has a functional drug efflux system.

29. The method according to claim 18 or 19, wherein said bacterial strain is drug-resistant.

30. The method according to claim 29, wherein said bacterial strain is resistant to an antibiotic selected from the group consisting of: a fluoroquinolone antibiotic, a β-lactam antibiotic, an aminoglycoside antibiotic, and a macrolide antibiotic.

31. The method according to claim 18 or 19, wherein said reporter gene is detectable by optical means.

32. The method according to claim 31, wherein said reporter gene is selected from the group consisting of: lacZ, gusA, cat, lux and gfp.

33. The method according to claim 31, wherein said reporter gene is detectable by fluorescence.

34. The method according to claim 33, wherein said reporter gene is gfp.

35. The method according to claim 31, wherein said method is performed in a high-throughput format.

36. The method according to claim 35, wherein said high-throughput format uses a 96-well plate or a 3456 NanoWell™ plate.
